(19) United States
(12) Patent Application Publication
(10) Pub. No.: US 2017/0296297 A1
(43) Pub. Date: Oct. 19, 2017

(54) WEIGHTED SURGICAL CLAMP

(71) Applicants: Joseph C. Zulty, Fallston, MD (US); David A. KOVALIK, Alexandria, VA (US)

(72) Inventors: Joseph C. Zulty, Fallston, MD (US); David A. KOVALIK, Alexandria, VA (US)

(21) Appl. No.: 15/486,857

(22) Filed: Apr. 13, 2017

Related U.S. Application Data
(60) Provisional application No. 62/322,069, filed on Apr. 13, 2016.

(51) Int. Cl. A61B 90/50; F16B 2/10

(52) U.S. Cl. CPC A61B 90/50; F16B 2/10

(57) ABSTRACT

A weighted surgical clamp is provided for removable attachment to a surgical instrument. Embodiments include a weighted surgical clamp having a pair of opposing clamp arms pivoted relative to each other, and a pair of opposing grips disposed near respective ends of the clamp arms. The grips cooperate to immovably hold the surgical instrument relative to the weighted surgical clamp, and a spring or a ratchet lock is provided for positioning the grips such that they immovably attach the surgical clamp to the surgical instrument. A weight is included on one or both of the clamp arms for retaining the surgical instrument in a predetermined rotated position during a surgical procedure when the surgical instrument and the surgical clamp are attached to each other.

### RELATED APPLICATIONS

The present application claims priority to U.S. Provisional Application No. 62/322,069, entitled “Weighted Surgical Clamp,” filed Apr. 13, 2016, which is incorporated herein by reference in its entirety.

### FIELD

Embodiments relate generally to surgical instruments, and in particular to weighted clamps attachable to other surgical instruments.

### BACKGROUND

In a veterinary surgical ovariohysterectomy, a common surgical technique of the ventral abdominal approach includes the exteriorization and exposure of the ovarian pedicle, an anatomical collection of linear vessels and connective tissue often buried deep within a patient. Exposure of this anatomical bundle must be adequate for proper and complete ligation with removal of the ovary. Once exteriorized from the abdomen, this bundle is commonly clamped with between two to three conventional clamps or hemostats, such as Carmalts, before sutures are placed and the bundle is transected with a surgical blade. Once the hemostats have been placed, rotation of one of these clamps, typically 180 degrees (but sometimes between 45 and 270 degrees depending on technique and anatomy), can vastly improve bundle exposure and ease suture-placement and ligation. Disadvantageously, this rotation must be performed by an assistant or by dexterous maneuvering of the surgeon. Depending on the surgeon's preference, this technique with the instrument may also be applied in like manner to the uterus for its exposure and ligation.

There exists a need for an instrument to facilitate rotation of another instrument, such as a hemostat, and/or holding that rotation, during surgery.

### SUMMARY

One or more embodiments can include a weighted surgical clamp for removable attachment to a surgical instrument. The weighted surgical clamp comprises a pair of opposing clamp arms pivoted relative to each other, and a pair of opposing grips, each grip disposed proximal a first end of one of the clamp arms. The pair of grips is for cooperating with each other to immovably hold the surgical instrument relative to the weighted surgical clamp. A spring is provided for biasing the grips towards each other such that the grips immovably attach the surgical clamp to the surgical instrument; and a weight is disposed on one or both of the clamp arms for retaining the surgical instrument in a predetermined rotated position during a surgical procedure when the surgical instrument and the surgical clamp are attached to each other.

Embodiments can further include a weighted surgical clamp for removable attachment to a surgical instrument, the weighted surgical clamp comprising a pair of opposing clamp arms pivoted relative to each other, and a pair of opposing grips, each grip disposed proximal a first end of one of the clamp arms. The pair of grips is for cooperating with each other to immovably hold the surgical instrument relative to the weighted surgical clamp. A ratchet lock is provided for holding the grips in position relative to each other such that the grips immovably attach the surgical clamp to the surgical instrument; and a weight is disposed on one or both of the clamp arms for retaining the surgical instrument in a predetermined rotated position during a surgical procedure when the surgical instrument and the surgical clamp are attached to each other.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIGS. 1a and 1b are respectively side and top views of an instrument according to an embodiment of the present disclosure having a weight on one of its clamp arms.

FIGS. 1c and 1d are respectively perspective and side views of an instrument according to another embodiment of the present disclosure having a weight on one of its clamp arms.

FIGS. 1e and 1f are respectively perspective and side views of an instrument according to a further embodiment of the present disclosure having a weight integral with its clamp arms.

FIGS. 2a and 2b are respectively side and top views of an instrument according to an embodiment of the present disclosure having a sliding weight on one of its clamp arms.

FIG. 2c shows the instrument of FIGS. 2a-b disassembled for sterilization.

FIGS. 3a and 3b are respectively side and top views of an instrument according to an embodiment of the present disclosure having a pivoting weight, showing the weight pivoted inward.

FIG. 3c shows the instrument of FIGS. 3a-b with its weight pivoted outward.

FIGS. 4a and 4b are respectively side and top views of an instrument according to an embodiment of the present disclosure having detachable weights.

FIG. 4c shows the instrument of FIGS. 4a-b with a weight attached.

FIGS. 4d and 4e are respectively perspective and side views of an instrument according to another embodiment of the present disclosure having detachable weights.

FIG. 4f shows weights of differing masses for use with the instrument of FIGS. 4d-e.

FIG. 5 is a perspective view of a hemostat being held by the disclosed surgical instrument.

### DETAILED DESCRIPTION

It should be understood that the principles described herein are not limited in application to the details of construction or the arrangement of components set forth in the following description or illustrated in the following drawings. The principles can be embodied in other embodiments and can be practiced or carried out in various ways. Also, it is to be understood that the phraseology and terminology used herein is for the purpose of description and should not be regarded as limiting.

The instrument disclosed herein is a weighted clamp. In certain embodiments, when a conventional Carmalt has been placed on an ovarian bundle and rotated, the disclosed instrument clamps onto the Carmalt and holds its rotation. Alternatively, the disclosed instrument can be clamped to the Carmalt to facilitate and then hold its rotation. After the ovarian bundle has been transected, the disclosed instrument is removed and set aside, to be used again for the second ovary, and potentially for similar exposure of the uterine body for ligation and transection. While the disclosed instrument is described herein as being useful in the ovariohysterectomy of canine and feline patients, it should be understand that it can also be used for a variety of other procedures, surgical and otherwise.

Several embodiments of the novel instrument are disclosed herein. An advantageous feature of the instrument, in all its embodiments, is the ability to gently but firmly clamp and grip a Carmalt or other hemostat, and hold it in rotation as needed, as described in the aforementioned procedure. The entire instrument is constructed of surgery-grade materials as detailed herein below, and is accordingly durable and can be autoclaved. The disclosed instrument can also be handled ambidextrously and, in all embodiments, typically with just one hand.

The disclosed instrument is a clamp having a pair of clamp arms that rotate relative to each other on a pivot. The clamp arms each have a grip. The pair of grips cooperate with each other to hold a surgical instrument such as a hemostat, and are secured in position by a ratchet-lock, or biased in position by a spring. In certain embodiments, one clamp arm terminates in a handle for the surgeon's second or third finger. The other clamp arm terminates in a weight. Several embodiments of the weight are disclosed herein, including several mounting arrangements for the weight. The edges of the clamp arms that grasp the Carmalt or other surgical instrument are composed of a hard, durable elastic material such as a rubber, plastic, or silicone. The remainder of the instrument is stainless steel.

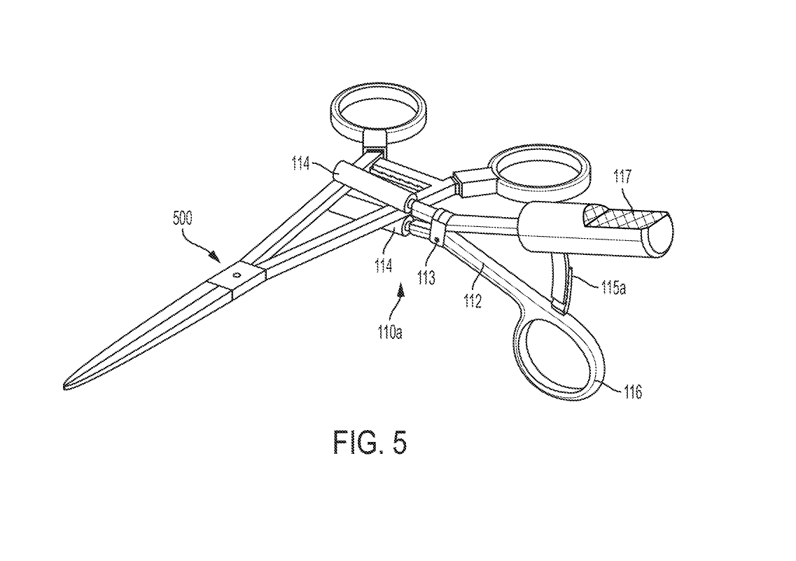

FIG. 5 shows a conventional hemostat 500 being held by an embodiment of the disclosed weighted surgical clamp 110a, which is described in detail herein below. Although FIG. 5 illustrates hemostat 500 being immovably held by a particular embodiment of the disclosed weighted clamp, those of skill in the art will understand that all embodiments of the disclosed weighted clamp described herein below are for immovably holding a conventional surgical instrument (such as a hemostat) between a pair of grips, in a manner similar to that shown in FIG. 5.

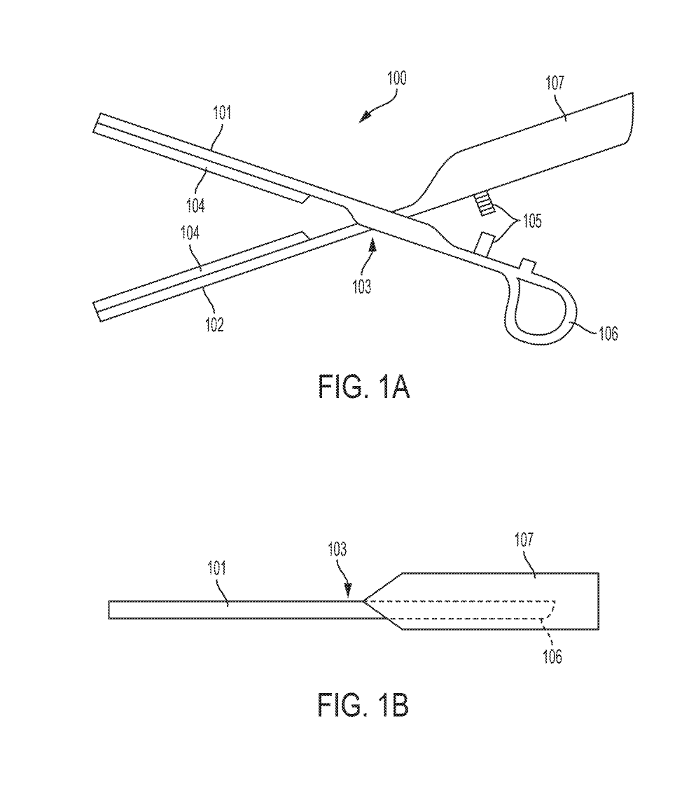

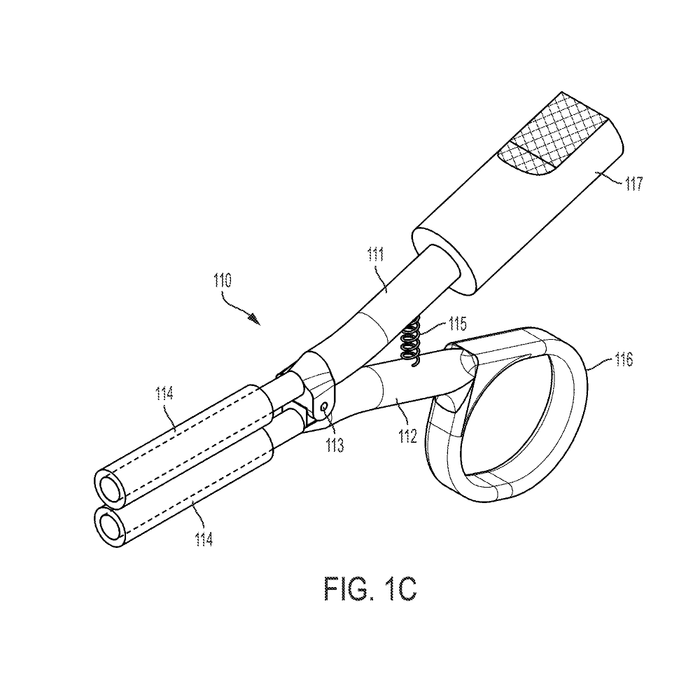

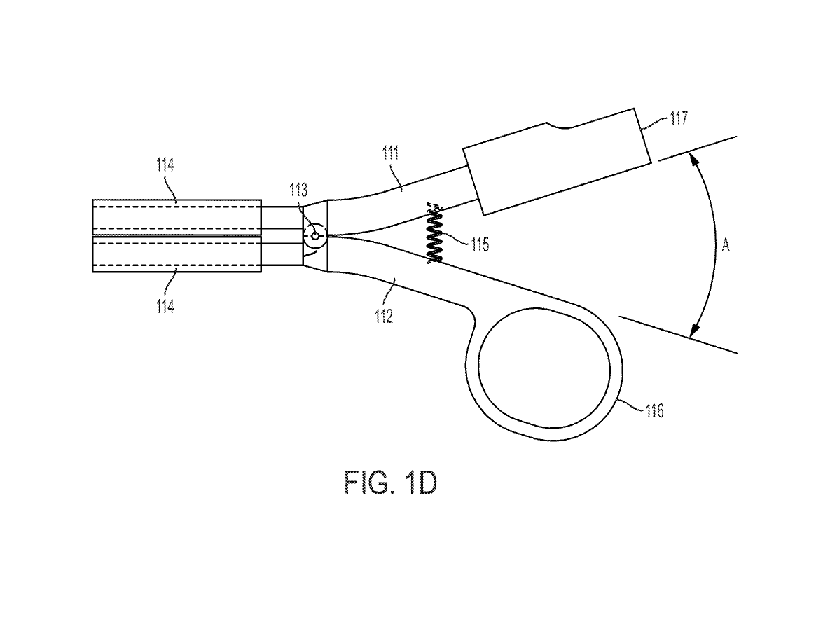

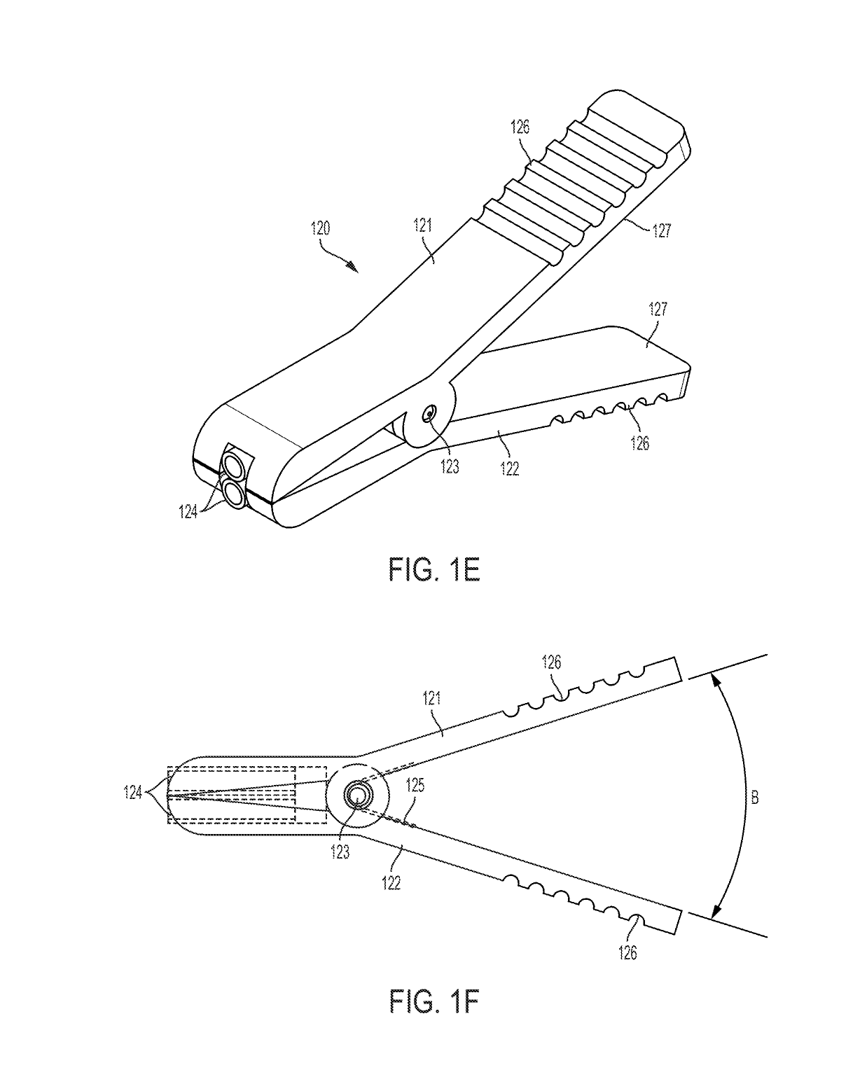

In an exemplary embodiment shown in FIGS. 1a-1b of a disclosed weighted surgical clamp 100, two opposing clamp arms 101,102 are pivoted relative to each other at a pivot 103. A pair of opposing grips 104 of an elastic material, such as rubber, plastic, or silicone, is attached to the clamp arms 101, 102 near the end of the clamp arms 101-102. Grips 104 are for cooperating with each other to immovably hold a surgical instrument such as a hemostat (not shown) relative to the weighted surgical clamp 100, as by engaging a handle of the surgical instrument as shown in FIG. 5. A conventional ratchet lock 105 holds the grips 104 in position relative to each other such that grips 104 immovably attach the surgical clamp 100 to the surgical instrument without damaging the surgical instrument. One of the clamp arms 101 terminates at a handle 106, and the terminus of the other clamp arm 102 is a simple weight 107; for example, approximately 10-15 cm. long and having a mass of approximately 100-250 grams. In this embodiment, the weight 107 is integral with the clamp arm 102. Weight 107 is for retaining the surgical instrument in a predetermined rotated position during a surgical procedure when the surgical instrument and the surgical clamp 100 are attached to each other. Advantages of this embodiment include its simplicity, and the ability to be placed and removed with one hand.

In a further embodiment shown in FIGS. 1c-d, instead of a ratchet lock, the clamp arms of the weighted surgical clamp are biased towards each other by a spring. In this embodiment, weighted clamp 110 comprises a pair of opposing clamp arms 111, 112 pivoted relative to each other at a pivot 113. A pair of opposing grips 114 of an elastic material, such as rubber, plastic, or silicone, is attached to the clamp arms 111, 112 near the end of the clamp arms 111, 112. Grips 114 are for cooperating with each other to immovably hold a surgical instrument (not shown) relative to the weighted surgical clamp 110, as by engaging a handle of the surgical instrument. A spring 115, such as a coil spring (as shown) or a leaf spring, is provided between clamp arms 111, 112 for biasing the grips 114 towards each other such that the grips 114 immovably attach the surgical clamp 110 to the surgical instrument without damaging the surgical instrument. One of the clamp arms 112 terminates at a handle 116, and the terminus of the other clamp arm 111 is a simple weight 117. In this embodiment, the weight 117 is integral with the clamp arm 111. Weight 117 is for retaining the surgical instrument in a predetermined rotated position during a surgical procedure when the surgical instrument and the surgical clamp 110 are attached to each other. FIG. 1d shows exemplary dimensions for several parts of clamp 110. For example, for ease of use, clamp arms 111, 112 are arranged to form an angle A of about 35 degrees between handle 116 and weight 117 when grips 114 are parallel to each other.

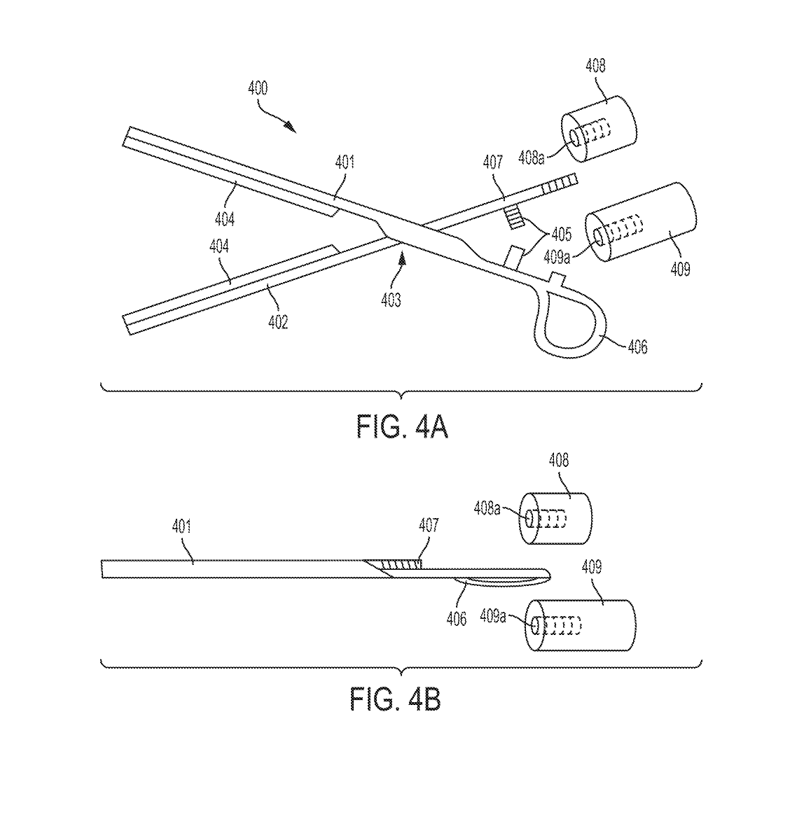

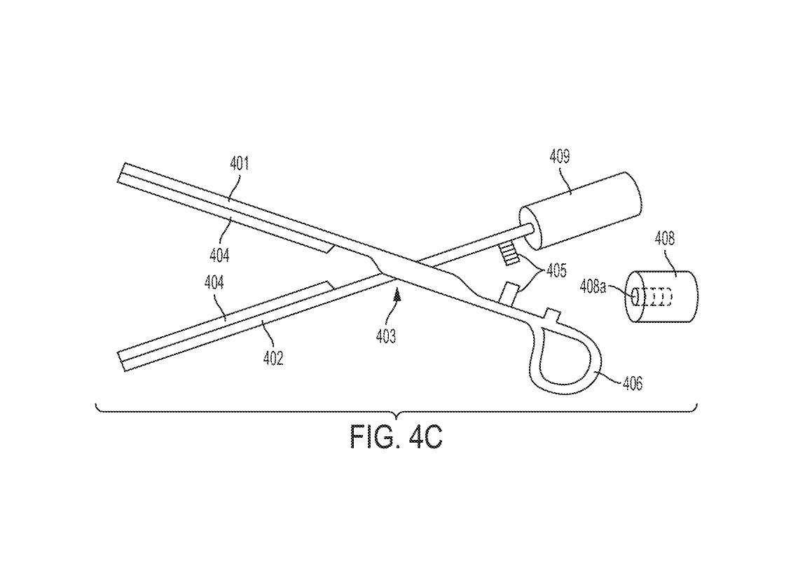

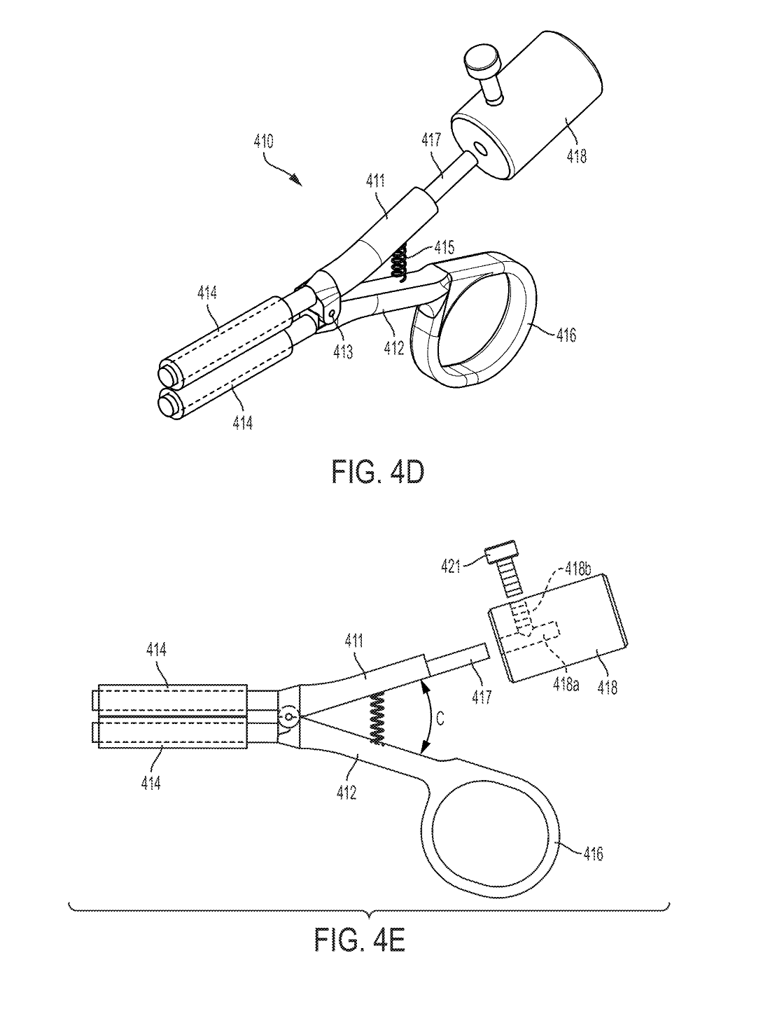

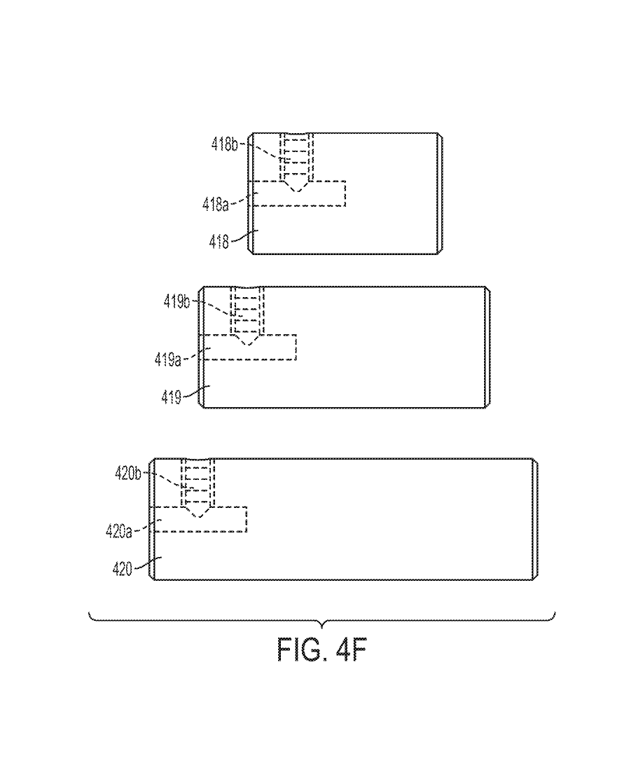

FIG. 5 illustrates a slightly different embodiment of the weighted clamp 110 of the embodiment of FIGS. 1c-d. In this embodiment, a weighted clamp 110a has a conventional ratchet lock 115a for holding grips 114 in position to immovably hold hemostat 500, instead of a spring. Clamp 110a is otherwise identical to clamp 110. FIG. 5 shows weighted clamp 110a holding a conventional hemostat 500 between its grips 114. Those of skill in the art will understand that all the embodiments described herein featuring a spring to bias the pair of grips toward each other (e.g., the embodiments of FIGS. 1c-d, FIGS. 1e-f, and FIGS. 4d-f) could have a conventional ratchet lock instead of a spring, as shown in FIG. 5.

In a similar embodiment shown in FIGS. 1e-f, the weight of the weighted surgical clamp is integrated into both clamp arms. In this embodiment, weighted clamp 120 comprises a pair of opposing clamp arms 121, 122 pivoted relative to each other at a pivot 123. A pair of opposing grips 124 of an elastic material, such as rubber, plastic, or silicone, is attached to the clamp arms 121, 122 near the end of the clamp arms 121, 122, for cooperating with each other to immovably hold a surgical instrument (not shown) relative to the weighted surgical clamp 120, as by engaging a handle of the surgical instrument. A spring 125, such as a leaf spring or a coil spring, is provided for biasing the grips 124 towards each other such that the grips 124 immovably attach the surgical clamp 120 to the surgical instrument without damaging the surgical instrument. Both of the clamp arms 121, 122 have ridges 126 or the like to allow easy gripping of clamp 120 by the fingers of the user. In this embodiment, a weight 127 is integral with both the clamp arms 121, 122. In other words, the aggregate weight of the clamp arms 121, 122 together is adequate for retaining the surgical instrument in a predetermined rotated position during a surgical procedure when the surgical instrument and the surgical clamp 120 are attached to each other. In certain embodiments, the total weight of surgical clamp 120 is between 150 and 200 grams. This total weight is generally suitable for any of the embodiments disclosed herein; the weighted end of the surgical clamp being heaviest. FIG. 1f shows exemplary dimensions for several parts of clamp 120. For example, for ease of use, clamp arms 121, 122 are arranged to form an angle B of about 35 degrees between clamp arms 121, 122 when grips 124 are parallel to each other.

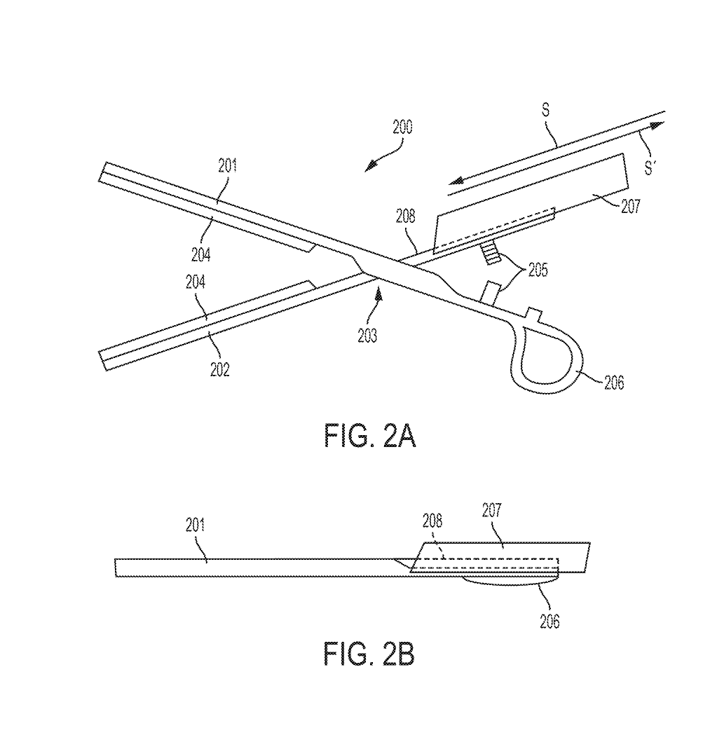

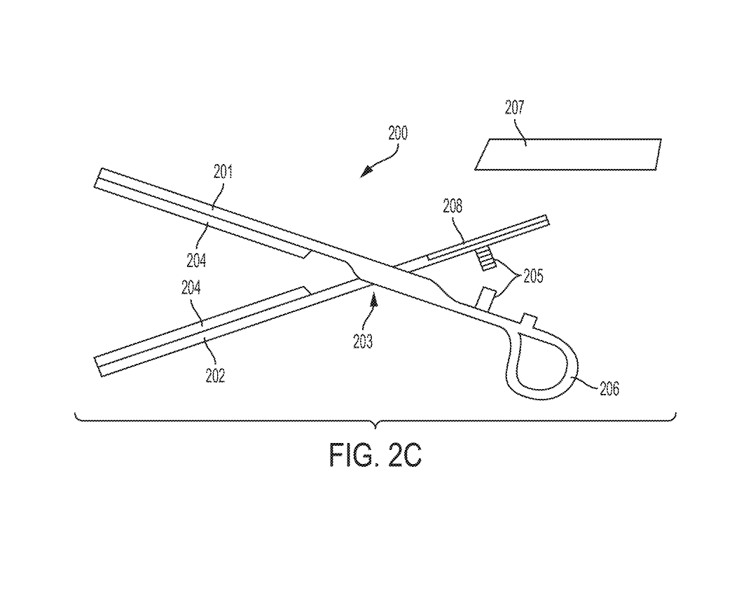

In certain other embodiments, the weight is movably attachable to an end of one of the clamp arms. In an exemplary embodiment shown in FIGS. 2a-2c of a disclosed weighted surgical clamp 200, two opposing clamp arms 201, 202 are pivoted relative to each other at a pivot 203, as in the embodiments of FIGS. 1a-f. A pair of opposing grips 204 of an elastic material, such as rubber, plastic, or silicone, is attached to the clamp arms 201, 202 near the end of the clamp arms 201-202. Grips 204 are for cooperating with each other to immovably hold a surgical instrument (not shown) relative to the weighted surgical clamp 200, as by engaging a handle of the surgical instrument. A conventional ratchet lock 205 holds the grips 204 in position relative to each other such that grips 204 immovably attach the surgical clamp 200 to the surgical instrument without damaging the surgical instrument.

One of the clamp arms 201 terminates at a handle 206. The terminus of the other clamp arm 202 is a detachable weighted slide 207 which is mounted to slide on a slide bed 208 along a portion of the length of clamp arm 202, in the directions of arrows S, S′. Leverage is maintained by adjusting the position of slide weight 207 on slide bed 208. The weighted slide 207 can be, for example, approximately 5-10 cm. long, and have a mass of about 100-250 grams. Slide weight 207 interlocks with the slide bed 208 of clamp arm 202 to retain the slide weight 207 at a slide position set by a user. Those of skill in the art will appreciate that the interlock between slide weight 207 and slide bed 208 can be accomplished via friction between slide weight 207 and slide bed 208 in a conventional manner, as by selectively sizing the respective parts. The slide weight 207 can be separated from the rest of the clamp 200 before autoclaving, for thorough cleaning. Advantages of this embodiment include greater versatility concerning its leverage.

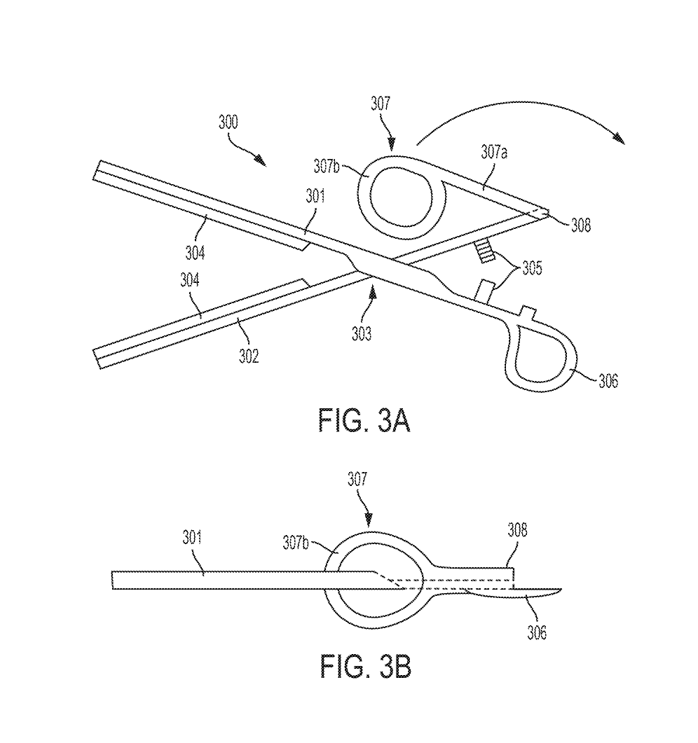

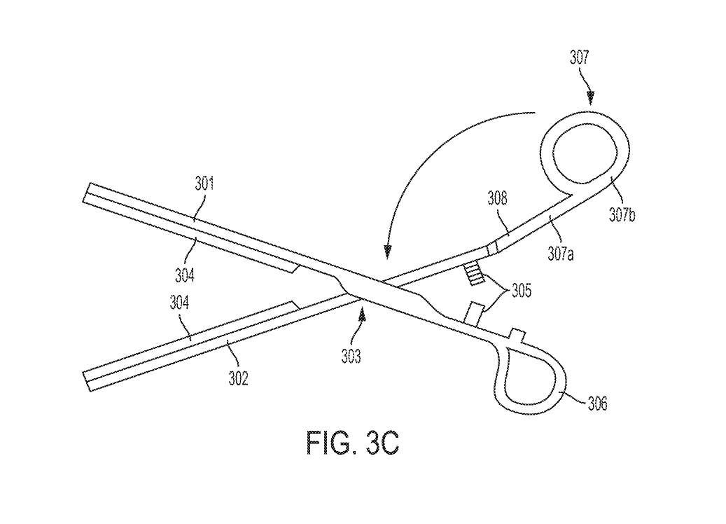

According to a further exemplary embodiment, shown in FIGS. 3a-3c, a disclosed weighted surgical clamp 300 has a pivotably mounted rotating weight. Two opposing clamp arms 301, 302 are pivoted relative to each other at a pivot 303, as in the embodiments of FIGS. 1a-f. A pair of opposing grips 304 of an elastic material, such as rubber, plastic, or silicone, is attached to the clamp arms 301, 302 near the end of the clamp arms 301-302. Grips 304 are for cooperating with each other to immovably hold a surgical instrument (not shown) relative to the weighted surgical clamp 300, as by engaging a handle of the surgical instrument. A conventional ratchet lock 305 holds the grips 304 in position relative to each other such that grips 304 immovably attach the surgical clamp 300 to the surgical instrument without damaging the surgical instrument.

One of the clamp arms 301 terminates at a handle 306. The terminus of the other clamp arm 302 is a pivotably mounted weight 307, such as a spherically-shaped weight, attached to a pivot 308 near the end of claim arm 302. Leverage is maintained by swinging weight 307 outward on pivot 308. Once positioned at an appropriate angle, the weight 307 is fastened by tension on the pivot 308. In the embodiment of FIGS. 3a-c, weight 307 comprises a slender pivot arm 307a and a bulbous portion 307b at a first end of the arm 307a. Bulbous portion 307b can have a mass of, for example, approximately 100-250 grams. A second end of the arm 307a opposite the first end of the arm 307a is pivotably attached to pivot 308. Weight 307 is movable from a first position shown in FIGS. 3a and b, where the bulbous portion 307b is proximal to clamp arm 302, to a second position shown in FIG. 3c where the bulbous portion 307b is remote from clamp arm 302. Advantages of this embodiment include greater versatility concerning its leverage.

According to a further exemplary embodiment, shown in FIGS. 4a-4c, a disclosed weighted surgical clamp 400 has removable interchangeable weights. Two opposing clamp arms 401, 402 are pivoted relative to each other at a pivot 403, as in the embodiments of FIGS. 1a-f. A pair of opposing grips 404 of an elastic material, such as rubber, plastic, or silicone, is attached to the clamp arms 401, 402 near the end of the clamp arms 401-402. Grips 404 are for cooperating with each other to immovably hold a surgical instrument (not shown) relative to the weighted surgical clamp 400, as by engaging a handle of the surgical instrument. A conventional ratchet lock 405 holds the grips 404 in position relative to each other such that grips 404 immovably attach the surgical clamp 400 to the surgical instrument without damaging the surgical instrument.

One of the clamp arms 401 terminates at a handle 406. The terminus of the other clamp arm 402 is a threaded end portion 407. Weights 408, 409 of differing masses are provided with the instrument 400, each having a threaded hole or socket 408a, 409a engagable with threaded portion 407 of clamp arm 402. In use, one of the weights 408, 409 is secured to the instrument 400 to provide needed leverage. Weights 408, 409 can range from 100-250 grams.

In a further embodiment shown in FIGS. 4d-f, instead of a ratchet lock, the clamp arms of the weighted surgical clamp are biased towards each other by a spring (as in the embodiment of FIGS. 1c-d), and the clamp has removable interchangeable weights to function in the same manner as the embodiment of FIGS. 4a-c. In this embodiment, weighted clamp 410 comprises a pair of opposing clamp arms 411, 412 pivoted relative to each other at a pivot 413. A pair of opposing grips 414 of an elastic material, such as rubber, plastic, or silicone, is attached to the clamp arms 411, 412 near the end of the clamp arms 411, 412. Grips 414 are for cooperating with each other to immovably hold a surgical instrument (not shown) relative to the weighted surgical clamp 410, as by engaging a handle of the surgical instrument. A spring 415, such as a coil spring or a leaf spring, is provided for biasing the grips 414 towards each other such that the grips 414 immovably attach the surgical clamp 410 to the surgical instrument without damaging the surgical instrument.

One of the clamp arms 412 terminates at a handle 416, and the terminus of the other clamp arm 411 is a plain end portion 417. Weights 418, 419, 420 of differing masses are provided with the instrument 410, each having a hole 418a, 419a, 420a engagable with end portion 417 of clamp arm 412, and an intersecting threaded hole 418b, 419b, 420b for engaging the threads of a retaining bolt 421. In use, one of the weights 418, 419, 420 is secured to the instrument 410 via retaining bolt 421 to provide needed leverage. Weights 418, 419, 420 can range from 100-250 grams. FIG. 4e shows exemplary dimensions for several parts of clamp 410. For example, for ease of use, clamp arms 411, 412 are arranged to form an angle C of about 35 degrees between handle 416 and weight 418 when grips 414 are parallel to each other.

It is, therefore, apparent that there is provided in accordance with the present invention, a weighted surgical clamp. While this invention has been described in conjunction with a number of embodiments, it is evident that many alternatives, modifications and variations would be or are apparent to those of ordinary skill in the applicable arts. Accordingly, applicants intend to embrace all such alternatives, modifications, equivalents and variations that are within the spirit and scope of this invention.

What is claimed is:

1. A weighted surgical clamp for removable attachment to a surgical instrument, the weighted surgical clamp comprising:
a pair of opposing clamp arms pivoted relative to each other;
a pair of opposing grips, each grip disposed proximal a first end of one of the clamp arms, wherein the pair of grips is for cooperating with each other to immovably hold the surgical instrument relative to the weighted surgical clamp;
a spring for biasing the grips towards each other such that the grips immovably attach the surgical clamp to the surgical instrument; and
a weight disposed on one or both of the clamp arms for retaining the surgical instrument in a predetermined rotated position during a surgical procedure when the surgical instrument and the surgical clamp are attached to each other.

2. The surgical clamp of claim 1, wherein the grips comprise an elastic material for preventing damage to the surgical instrument when attached to the surgical clamp.

3. The surgical clamp of claim 2, wherein the grips are for engaging a handle of the surgical instrument.

4. The surgical clamp of claim 1, wherein the weight is integral with at least one of the clamp arms.

5. The surgical clamp of claim 1, wherein the weight is disposed proximal a second end of one of the clamp arms opposite the first end.

6. The surgical clamp of claim 5, wherein the weight is removably attachable to the second end of the one of the clamp arms.

7. The surgical clamp of claim 6, wherein the weight is mounted to slide along a portion of the length of the one of the clamp arms.

8. The surgical clamp of claim 5, wherein the weight is pivotably mounted proximal to the second end of the one of the clamp arms.

9. The surgical clamp of claim 5, comprising a handle for a user's finger at a second end of the other of the clamp arms, opposite the first end.

10. The surgical clamp of claim 6, comprising a plurality of the removable weights, each removable weight having a different mass, wherein exactly one of the removable weights is attachable to the second end of the one of the clamp arms at a time.

11. The surgical clamp of claim 7, wherein the one of the clamp arms has a slide bed for enabling the weight to slide, and the clamp further comprises an interlock between the slide bed and the weight to retain the weight at a slide position set by a user.

12. The surgical clamp of claim 8, wherein the weight comprises a slender pivot arm and a bulbous portion at a first end of the arm;
wherein a second end of the arm opposite the first end of the arm is pivotably attached proximal the second end of the one of the clamp arms; and
wherein the weight is movable from a first position where the bulbous portion is proximal to the one of the clamp arms, to a second position where the bulbous portion is remote from the one of the clamp arms.

13. A weighted surgical clamp for removable attachment to a surgical instrument, the weighted surgical clamp comprising:
a pair of opposing clamp arms pivoted relative to each other;
a pair of opposing grips, each grip disposed proximal a first end of one of the clamp arms, wherein the pair of grips is for cooperating with each other to immovably hold the surgical instrument relative to the weighted surgical clamp;
a ratchet lock for holding the grips in position relative to each other such that the grips immovably attach the surgical clamp to the surgical instrument; and
a weight disposed on one or both of the clamp arms for retaining the surgical instrument in a predetermined rotated position during a surgical procedure when the surgical instrument and the surgical clamp are attached to each other.

14. The surgical clamp of claim 13, wherein the grips comprise an elastic material for preventing damage to the surgical instrument when attached to the surgical clamp.

15. The surgical clamp of claim 14, wherein the grips are for engaging a handle of the surgical instrument.

16. The surgical clamp of claim 13, wherein the weight is integral with at least one of the clamp arms.

17. The surgical clamp of claim 13, wherein the weight is disposed proximal a second end of one of the clamp arms opposite the first end.

18. The surgical clamp of claim 17, wherein the weight is removably attachable to the second end of the one of the clamp arms.

19. The surgical clamp of claim 18, wherein the weight is mounted to slide along a portion of the length of the one of the clamp arms.

20. The surgical clamp of claim 17, wherein the weight is pivotably mounted proximal to the second end of the one of the clamp arms.

21. The surgical clamp of claim 17, comprising a handle for a user's finger at a second end of the other of the clamp arms, opposite the first end.

22. The surgical clamp of claim 18, comprising a plurality of the removable weights, each removable weight having a different mass, wherein exactly one of the removable weights is attachable to the second end of the one of the clamp arms at a time.

23. The surgical clamp of claim 19, wherein the one of the clamp arms has a slide bed for enabling the weight to slide, and the clamp further comprises an interlock between the slide bed and the weight to retain the weight at a slide position set by a user.

24. The surgical clamp of claim 20, wherein the weight comprises a slender pivot arm and a bulbous portion at a first end of the arm;
wherein a second end of the arm opposite the first end of the arm is pivotably attached proximal the second end of the one of the clamp arms; and
wherein the weight is movable from a first position where the bulbous portion is proximal to the one of the clamp arms, to a second position where the bulbous portion is remote from the one of the clamp arms.
